(19) United States
(12) Patent Application Publication
(10) Pub. No.: US 2018/0110491 A1
(43) Pub. Date: Apr. 26, 2018

(54) DYNAMIC IMAGE PROCESSOR

(71) Applicant: Konica Minolta, Inc., Tokyo (JP)

(72) Inventors: Shuta ISHIDA, Osaka (JP); Kenta SHIMAMURA, Tokyo (JP)

(21) Appl. No.: 15/784,677

(22) Filed: Oct. 16, 2017

(30) Foreign Application Priority Data
Oct. 25, 2016 (JP) 2016-208447

(51) Int. Cl. A61B 6/00; A61B 6/03

(52) U.S. Cl. CPC A61B 6/463; G06T 7/0012; A61B 6/5241; A61B 6/037

(57) ABSTRACT

A dynamic image processor which measures a position of a structure in a plurality of frame images obtained by dynamic imaging, the dynamic image processor including a hardware processor which calculates an evaluation value indicating similarity to the structure for each position in each of the frame images, extracts at least one position candidate of the structure from each of the frame images based on the evaluation value, extracts a plurality of position candidates from at least one of the frame images, stores a plurality of route candidates in a storage by chronologically linking the position candidate extracted from each of the frame images to be a route candidate, determines one of the route candidates as a movement route of the structure, and determines the position candidate included in the determined route as the position of the structure in each of the frame images.

### BACKGROUND

### 1. Technological Field

The present invention relates to a dynamic image processor.

### 2. Description of the Related Art

The recent development in flat panel detectors (FPDs) for X-ray moving images has enabled the capturing of radiographic dynamic images. Computed tomography (CT) systems and magnetic resonance imaging (MRI) systems are expensive and thus have been installed in only limited medical institutions. In contrast, radiographic dynamic imaging systems are relatively not expensive and can be installed in many medical institutions. Through the use of such radiographic dynamic imaging systems, doctors can easily observe movement of structures inside living bodies and make diagnoses.

An extraction process using template matching is known as a method for extracting the position of a predetermined structure in a living body from a medical image. In the template matching, a reference image of a target structure to be extracted is prepared as a template image, the template image is moved over a target image, and a correlation values are calculated for areas in the target image overlapping the template image. Usually, the area having the maximum correlation value is extracted to be the position of the target structure.

For example, Japanese Patent Application Laid-Open Publication No. 2014-76331 discloses a technique using template matching for calculation of movement of the heart wall of a subject in a three-dimensional ultrasonic moving image.

Japanese Patent No. 5667489 discloses a technique of template matching for searching the position of a marker implanted near a tumor in a living body in a two-dimensional x-ray transparent image and identifying the position of the tumor from the positional relationship between the tumor and the marker.

In Japanese Patent Application Laid-Open Publication No. 2014-76331, template matching is applied to three-dimensional images, as described above. Unfortunately, in a two-dimensional radiographic dynamic image, structures overlap, causing blurring of the contours of the structures. The template matching applied to such a two-dimensional dynamic image leads to low correlation values of the positions of the target structure and may preclude the accurate detection of the actual position of the target structure in some cases. FIG. 11 illustrates an example radiographic dynamic image. The counter of the diaphragm is clear in the image on the left in FIG. 11, whereas the contour in the image on the right is unclear because of overlapping of the heart and ribs with the diaphragm.

In Japanese Patent No. 5667489, the position and movement of a spherical marker implanted inside a living body in a x-ray transparent image is observed by template matching. Unfortunately, this technique can only be used at limited facilities because the use of markers increases costs and requires complicated medical techniques. In the template matching without a marker on an organ, such as a diaphragm, selected as a target structure, the organ or diaphragm appears less clearly in an image compared to a marker and deforms over time. Thus, detection of the position based on extraction of a position corresponding to the maximum correlation value is not highly accurate.

### SUMMARY

An object of the present invention is to accurately determine the position of a target structure in frame images of a dynamic image obtained by radiographic image capturing of a subject without a marker even if the contour of the target structure is unclear.

To achieve at least one of the abovementioned objects, according to an aspect of the present invention, a dynamic image processor reflecting one aspect of the present invention measures a position of a predetermined structure in a plurality of frame images obtained by emitting radiation to a subject to perform dynamic imaging, and the dynamic image processor includes a hardware processor which calculates an evaluation value indicating similarity with respect to the structure for each position in a predetermined range in each of the plurality of frame images, extracts at least one position candidate of the structure from each of the plurality of frame images based on the calculated evaluation value, extracts a plurality of position candidates of the structure from at least one of the frame images, stores a plurality of route candidates in a storage, each of the route candidates being obtained by chronologically linking the position candidate of the structure extracted from each of the plurality of frame images to be a route candidate of movement of the structure, determines one of the plurality of route candidates stored in the storage as a movement route of the structure, and determines the position candidate of the structure included in the determined route as the position of the structure in each of the plurality of frame images.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 illustrates the overall configuration of a position measurement system according to an embodiment of the present invention;

FIG. 2 is a flow chart illustrating an image capturing control process carried out by a controller of an image capturing console in FIG. 1;

FIG. 3 is a flow chart illustrating the position measurement process carried out by the controller of the image capturing console in FIG. 1;

FIG. 4 illustrates an example initial setting input screen;

FIG. 5 illustrates a technique of extracting diaphragm position candidates;

FIG. 6 illustrates example storage data items in a route storage unit;

FIG. 7 illustrates a process of limiting route candidates;

FIG. 8 illustrates an example measurement result screen;

FIG. 9 illustrates an example result correction screen;

FIG. 10 illustrates another example result correction screen; and

FIG. 11 illustrates the contour of a diaphragm in a dynamic image of a chest area.

### DETAILED DESCRIPTION OF EMBODIMENTS

Hereinafter, one or more embodiments of the present invention will be described in detail with reference to the drawings. However, the scope of the invention is not limited to the disclosed embodiments or illustrated examples.

### [Configuration of Position Measurement System 100]

The configuration will now be described.

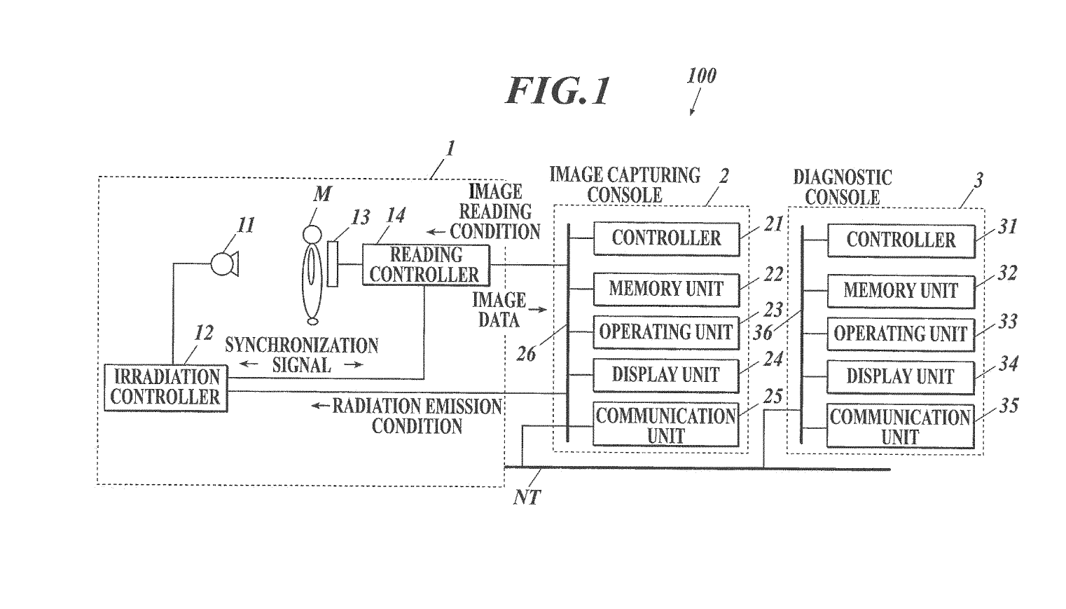

FIG. 1 illustrates the overall configuration of a position measurement system 100 according to an embodiment.

With reference to FIG. 1, the position measurement system 100 includes an image capturing device 1, an image capturing console 2 connected to the image capturing device 1 through a communication cable, and a diagnostic console 3 connected to the image capturing console 2 via a communication network NT, such as a local area network (LAN). The components of the position measurement system 100 meet the digital image and communications in medicine (DICOM) standard and communicate with each other in accordance with the DICOM.

### [Configuration of Image Capturing Device 1]

The image capturing device 1 is an image capturing unit that captures a dynamic state of a living body, such as expansion and contraction of a lung due to respiratory movement or pulsation of a heart. In dynamic imaging, a plurality of images is captured through repeated irradiation of a subject with pulsed radiation rays such as X rays at predetermined time intervals (pulsed radiation) or uninterrupted irradiation with low-dosage radiation (continuous radiation). The series of images captured through dynamic imaging is collectively referred to as a dynamic image. A dynamic image consists of a plurality of frame images. In this embodiment described below, dynamic imaging is performed through pulsed radiation. In this embodiment described below, the target site is the chest area. It should be noted that any other site may be a target site.

A radiation source 11 faces a radiation detector 13 with a subject M disposed therebetween. The radiation source 11 emits radiation rays (X-rays) toward the subject M under control of an irradiation controller 12.

The irradiation controller 12 is connected to the image capturing console 2 and controls the radiation source 11 on the basis of a radiation emission condition input from the image capturing console 2, to perform radiographic image capturing. The radiation emission condition input from the image capturing console 2 includes, for example, pulse rate, pulse width, pulse interval, the number of frames captured during a single image capturing operation, the value of the current flowing in the X-ray tube, the value of the voltage of the X-ray tube, or the type of an additional filter. The pulse rate is the number of radiation emission pulses per second and is equal to the frame rate described below. The pulse width is the radiation emission period per radiation emission. The pulse interval is the time from the start of a radiation emission to the start of the next radiation emission and is equal to the frame rate described below.

The radiation detector 13 includes a semiconductor image sensor, such as an FPD. An FPD includes, for example, a glass substrate, and a matrix of a plurality of detecting elements (pixels) disposed on the glass substrate at a predetermined position. The pixels detect the radiation rays according to the intensities emitted from the radiation source 11 and passing through at least the subject M, convert the detected radiation rays into electric signals, and store the electric signals. The pixels include switching units, such as thin film transistors (TFTs). The FPDs that can be used in this embodiment are classified into indirect FPDs and direct FPDs. An indirect FPD converts the detected X rays into electric signals at photoelectric transducers via a scintillator, whereas a direct FPD directly converts the detected X rays into electric signals.

The radiation detector 13 faces the radiation source 11 and the subject M is disposed therebetween.

A reading controller 14 is connected to the image capturing console 2. The reading controller 14 controls the switching unit of each pixel of the radiation detector 13 on the basis of an image reading condition from the image capturing console 2, to switch the reading of the electrical signals stored in each pixel and read the electrical signals stored in the radiation detector 13, to acquire image data. This image data corresponds to a frame image. The reading controller 14 then outputs the acquired frame image to the image capturing console 2. The image reading condition includes, for example, the frame rate, the frame interval, the pixel size, or the image size (matrix size). The frame rate is the number of frame images acquired per second and is equal to the pulse rate. The frame interval is the time from the start of an operation of acquiring a frame image to the start of the next operation of acquiring the next frame image and is equal to the pulse rate.

The irradiation controller 12 and the reading controller 14 are connected to each other and communicate synchronization signals, to synchronize the operations of radiation emission and image reading.

### [Configuration of Image Capturing Console 2]

The image capturing console 2 outputs a radiation emission condition and an image reading condition to the image capturing device 1 to control the capturing of radiographic images and the reading of radiographic images by the image capturing device 1. The image capturing console 2 also displays the dynamic image acquired by the image capturing device 1 to allow confirmation by the operator, such as a radiologist, on whether the image is suitable for confirmation of positioning and diagnosis.

The image capturing console 2 includes a controller 21, a memory unit 22, an operating unit 23, a display unit 24, and a communication unit 25, which are connected via a bus 26, as illustrated in FIG. 1.

The controller 21 includes a central processing unit (CPU) and a random access memory (RAM). The CPU of the controller 21 reads the system program and various processing programs stored in the memory unit 22 in response to operation of the operating unit 23, loads the read programs to the RAM, and executes various processes, such as the image capturing control process described below, in accordance with the loaded programs, to comprehensively control the operation of the components of the image capturing console 2 and the radiation emission and image reading by the image capturing device 1.

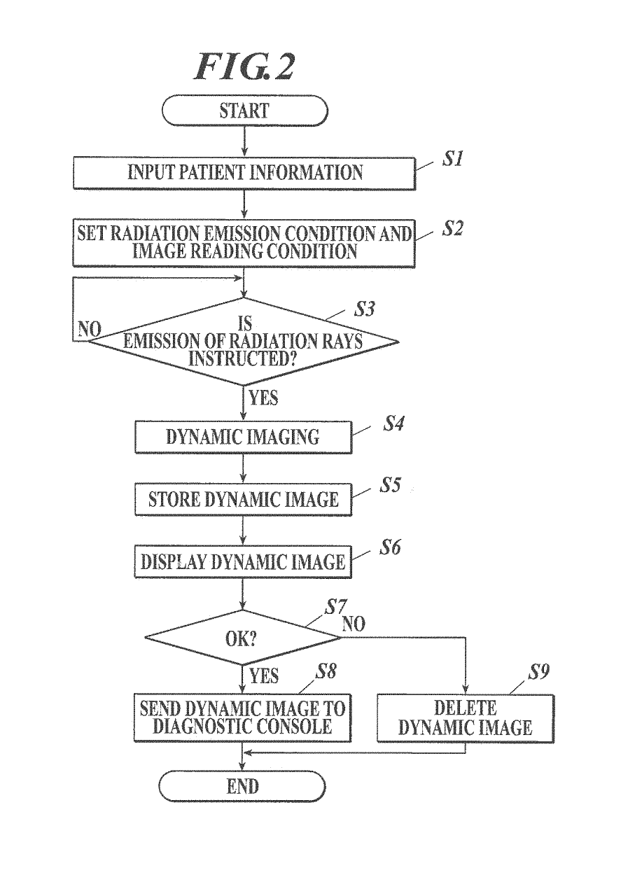

The memory unit 22 includes a non-volatile semiconductor memory or a hard disk. The memory unit 22 stores various programs to be executed by the controller 21, parameters required for the execution of the programs, and data on the results of the processes. For example, the memory unit 22 stores a program for executing the image capturing control process illustrated in FIG. 2. The memory unit 22 also stores radiation emission conditions and image reading conditions in correlation with target sites. These programs are stored in the form of readable program codes. The controller 21 sequentially carries out operations in accordance with the program codes.

The operating unit 23 includes a keyboard including cursor keys, numeral input keys, and various function keys and a pointing device, such as a mouse. The operating unit 23 outputs, to the controller 21, instruction signals input by key operation of the keyboard or mouse operation. The operating unit 23 may further include a touch panel on the display screen of the display unit 24. In such a case, instruction signals input via the touch panel are output to the controller 21.

The display unit 24 includes a monitor, such as a liquid crystal display (LCD) or a cathode ray tube (CRT). The display unit 24 displays input instructions from the operating unit 23 and various data in accordance with the instruction of display signals input from the controller 21.

The communication unit 25 includes a LAN adapter, a modem, and a terminal adapter (TA). The communication unit 25 controls the transmission and reception of data with components connected to the communication network NT.

### [Configuration of Diagnostic Console 3]

The diagnostic console 3 is a dynamic image processor that supports diagnoses by doctors through analysis of dynamic images sent from the image capturing console 2.

The diagnostic console 3 includes a controller 31, a memory unit 32, an operating unit 33, a display unit 34, and a communication unit 35, which are connected via a bus 36, as illustrated in FIG. 1.

The controller 31 includes a CPU, a RAM and such like. The CPU of the controller 31 reads the system program and various processing programs stored in the memory unit 32 in response to operation of the operating unit 33, loads the read programs to the RAM, and executes various processes, such as the position measurement process described below, in accordance with the loaded programs, to comprehensively control the operation of the components of the diagnostic console 3.

The memory unit 32 includes a non-volatile semiconductor memory, a hard disk or the like. The memory unit 32 stores various programs, such as a program executed by the controller 31 to carry out position measurement process, parameters required for the execution of the programs, and data on the results of the processes. These programs are stored in the form of readable program codes. The controller 31 sequentially carries out operations in accordance with the program codes.

The memory unit 32 stores the dynamic images from the image capturing console 2 in correlation with the position measurement results of the dynamic images.

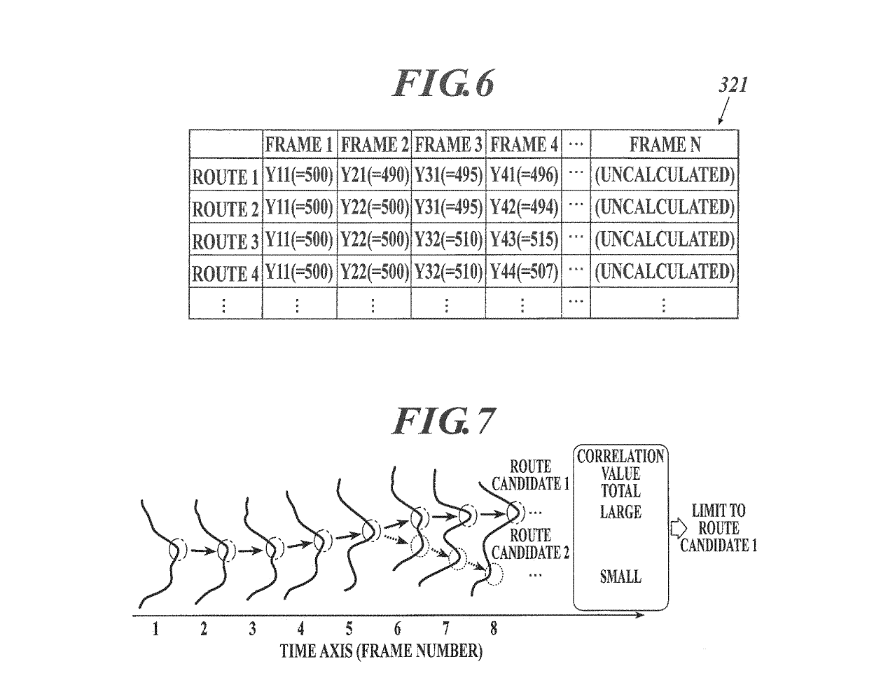

The memory unit 32 includes a route storage unit 321 that stores route candidates of the position measurement process (see FIG. 6). The route storage unit 321 will be described in detail below.

The operating unit 33 includes a keyboard including cursor keys, numeral input keys, and various function keys and a pointing device, such as a mouse. The operating unit 33 outputs, to the controller 31, instruction signals input by key operation of the keyboard or mouse operation. The operating unit 33 may further include a touch panel on the display screen of the display unit 34. In such a case, instruction signals input by touching the touch panel with a finger or a touch pen are sent to the controller 31.

The display unit 34 includes a monitor, such as an LCD or a CRT. The display unit 34 performs various types of display in accordance with instruction of display signals input via the controller 31.

The communication unit 35 includes a LAN adapter, a modem, and a TA. The communication unit 35 controls the transmission and reception of data with components connected to the communication network NT.

### [Operation of Position Measurement System 100]

The operation of the position measurement system 100 will now be described.

### (Operations of Image Capturing Device 1 and Image Capturing Console 2)

The image capturing operations of the image capturing device 1 and the image capturing console 2 will now be described.

FIG. 2 illustrates the image capturing control process carried out by the controller 21 of the image capturing console 2. The image capturing control process is carried out in cooperation of the controller 21 and the program stored in the memory unit 22.

The operator operates the operating unit 23 of the image capturing console 2 to input patient information (for example, name, height, weight, age, and sex) on a subject being tested (subject M) and examination information (on a target site (chest area in this case)) (step S1).

The radiation emission condition is read from the memory unit 22 and set in the irradiation controller 12, and the image reading condition is read from the memory unit 22 and set in the reading controller 14 (step S2).

The operating unit 23 waits for the instruction for emission of radiation rays (step S3). The operator disposes the subject M between the radiation source 11 and the radiation detector 13 and carries out positioning. Images in this embodiment are captured while the subject being tested (subject M) is breathing. Thus, the operator instructs the subject to relax and breathe quietly. Alternatively, the operator may induce deep breathing of the subject through verbal instructions, such as “inhale, exhale.” Upon completion of preparation of image capturing, the operating unit 23 is operated to input an instruction for emission of radiation rays.

Upon input of the instruction for emission of radiation rays at the operating unit 23 (YES in step S3), an image capturing start instruction is output to the irradiation controller 12 and the reading controller 14, to start dynamic imaging (step S4). In detail, the radiation source 11 emits radiation rays in pulse intervals determined by the irradiation controller 12, and the radiation detector 13 captures frame images.

Upon completion of image capturing of a predetermined number of frames, the controller 21 outputs an instruction for ending the image capturing to the irradiation controller 12 and the reading controller 14, to stop the image capturing operation. The number of frames to be captured should be at least enough for capturing a single respiratory cycle.

The captured frame images are sequentially input to the image capturing console 2, connected to numbers indicating the order of image capturing (frame number), stored in the memory unit 22 (step S5), and displayed on the display unit 24 (step S6). The operator confirms the positioning by observing the displayed dynamic image and determines whether an image suitable for diagnosis has been captured (image capturing OK) or recapturing is necessary (image capturing NG). The determined result is input by the operation of the operating unit 23.

Upon input of the determined result indicating “image capturing OK” through a predetermined operation of the operating unit 23 (YES in step S7), information such as an ID for identifying the dynamic image, patient information, examination information, a radiation emission condition, an image reading condition, and a number indicating the order of image capturing (frame number) are added to each frame image in the series of frame images captured through dynamic imaging (for example, the information is written in the header area of the image data in accordance with the DICOM). The frame images are then sent to the diagnostic console 3 via the communication unit 25 (step S8). The process then ends. In contrast, upon input of the determined result indicating “image capturing NG” through a predetermined operation of the operating unit 23 (NO in step S7), the series of frame images stored in the memory unit 22 are deleted (step S9), and the process ends. In this case, frame images must be recaptured.

It is preferred that the image capturing console 2 acquire information on the respiratory movement of the subject being tested (for example, waveforms indicating the respiratory movement) from a respiratory sensor worn by the subject being tested and in synchronization with the dynamic imaging during capturing of a dynamic image, on the basis of the acquired information, determine the information on the respiratory movement during the dynamic imaging (for example, the number of breaths, the times of the expiratory interval and inspiratory interval per breath, and the respiratory condition at each timing of frame image capturing (for example, expiratory interval, resting (maximal) expiratory position, inspiratory interval, and resting (maximal) inspiratory position)) by the controller 21, and add the determination result to each frame image.

### (Operation of Diagnostic Console 3)

The operation of the diagnostic console 3 will now be described.

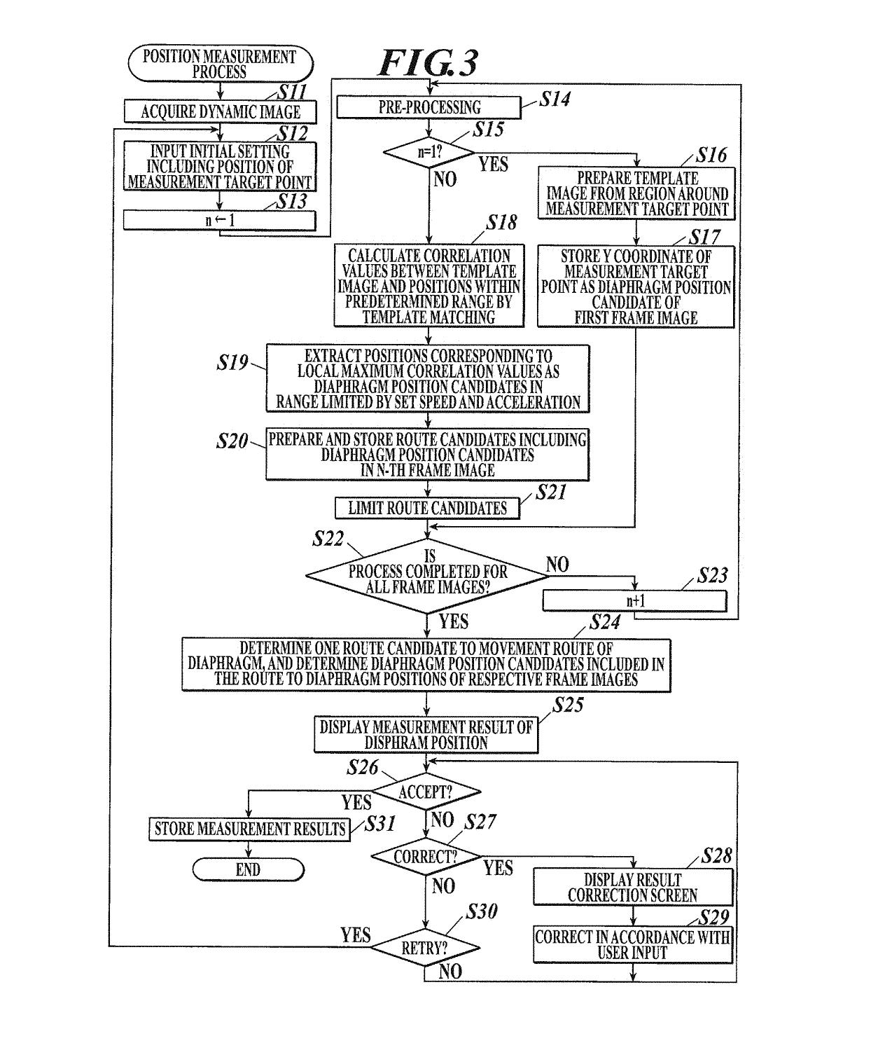

The diagnostic console 3 receives a series of frame images of dynamic images from the image capturing console 2 via the communication unit 35 and stores the series of frame images of the dynamic images in the memory unit 32. The operating unit 33 selects a dynamic image among the dynamic images stored in the memory unit 32, and the execution of the position measurement of a structure is instructed. In response, the position measurement process illustrated in FIG. 3 is carried out on the selected dynamic image through cooperation of the controller 31 and the program stored in the memory unit 32. Variations of the steps of the position measurement process and the functions of the screens are also achieved through cooperation of the controller 31 and the programs stored in the memory unit 32.

In the position measurement process according to this embodiment, the user specifies a position of a structure which is the position measurement target (referred to as a measurement target point) in a frame image (which is the first frame image in this embodiment), and a position of the structure is measured through template matching with a template image which is the image of the specified position. This embodiment will now be described through an example process of position measurement of a diaphragm.

The flow of the position measurement process will now be described with reference to FIG. 3.

The selected dynamic image is retrieved from the memory unit 32 (step S11).

An initial setting input screen 341 is displayed on the display unit 34 and receives input from a user through operation of the operating unit 33 on the initial setting including the position of the measurement target point (step S12: initial setting input unit).

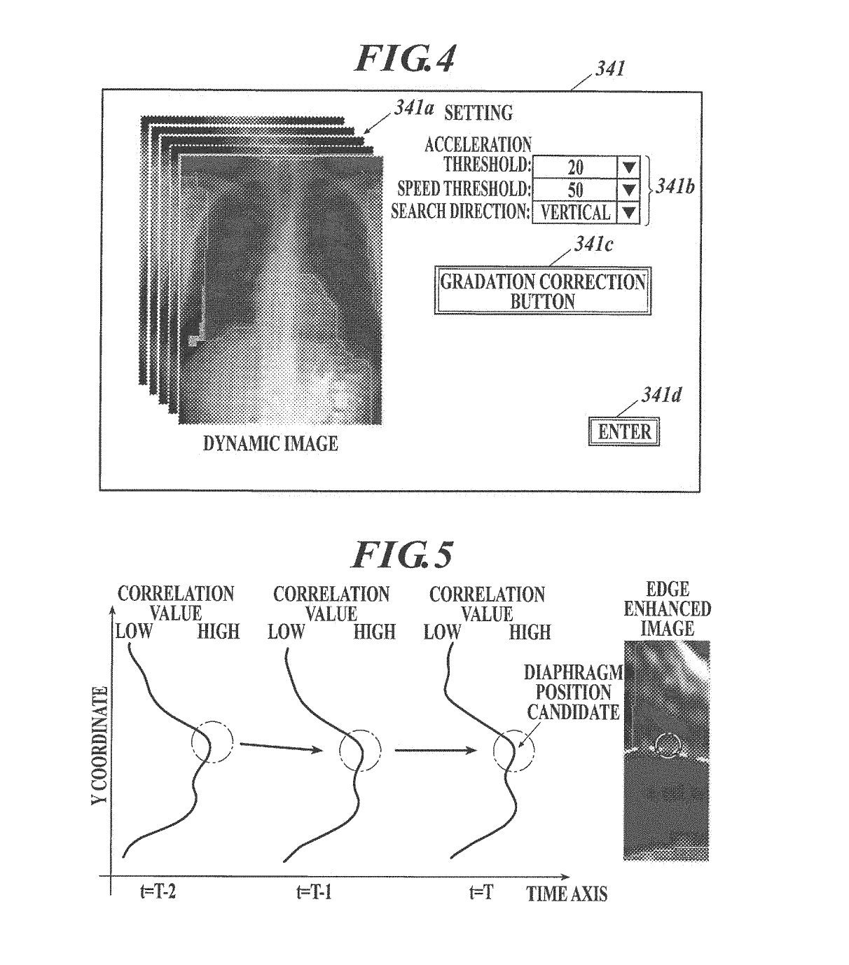

FIG. 4 illustrates an example initial setting input screen 341. The initial setting input screen 341 receives input from the user on information required for preparation of a template image for position measurement and setting of the extraction range of diaphragm position candidates. With reference to FIG. 4, the initial setting input screen 341 includes an image display region 341a, an initial setting input region 341b that receives an input of a parameter required for setting the extraction range of the diaphragm position candidates, a gradation correction button 341c, and an enter button 341d.

The image display region 341a displays the first frame image of the dynamic image. The user can operate a pointing device, such as a touch pen or a mouse, of the operating unit 33 to specify a measurement target point on the frame image displayed in the image display region 341a. For example, the contour of the diaphragm in the frame image displayed in the image display region 341a is specified through operation (for example, clicking) of the pointing device of the operating unit 33, and the position of the specified point is stored in the RAM of the controller 31 as the measurement target point.

The gradation of the image displayed in the image display region 341a can be adjusted in response to a user operation. For example, the gradation of the image displayed in the image display region 341a is adjusted in response to the number of times the gradation correction button 341c is pressed via the operating unit 33. In this way, the user can specify a measurement target point in a state in which the diaphragm is most visible for the user. Alternatively, the controller 31 may carry out automatic gradation correction on the frame image displayed in the image display region 341a to display the gradation-corrected frame image in the image display region 341a and receive specification of the measurement target point.

In step S12 described above, the user manually specifies the measurement target point of the diaphragm. Alternatively, the controller 31 may carry out image processing on the displayed frame image to automatically recognize the position of the diaphragm and specify the measurement target point.

Any frame image selected by the user besides the first frame image may be used for specification of the measurement target point. For example, the user may manually select a desired frame image by viewing the frame images one by one. Alternatively, a user interface may be provided for the user to input the frame number of a desired frame image. In this way, the user can freely determine any measurement target point. For example, a pulldown menu for selection of the frame image to be displayed in the image display region 341a (for example, resting expiratory position, resting inspiratory position, an intermediate position between the resting expiratory position and the resting inspiratory position, and an intermediate position between the resting inspiratory position and the resting expiratory position) may be provided in the initial setting input screen 341 so that the user can select a desired frame image. The movement and shape of the diaphragm is in the most natural condition in the resting expiratory position. Thus, the frame image of the resting expiratory position may be automatically displayed in the image display region 341a so that the user can specify the measurement target point.

It is preferred that the initial setting input screen 341 have a function of supporting the specification of a measurement target point by the user.

For example, a translucent image serving as a reference of the shape of the diaphragm may be overlaid on the frame image displayed in the image display region 341a. For example, the region of the diaphragm in the frame image of the resting expiratory position is identified, and an image indicating the shape of the identified diaphragm is overlaid on the frame image displayed in the image display region 341a. In this way, the measurement target point can be specified by the user after identifying an appropriate measurement target point representing the characteristics of the shape of the diaphragm.

In another scheme, the X coordinate of the measurement target point may be manually or automatically determined in advance, and the X coordinate is indicated by, for example, a line on the first frame image displayed in the image display region 341a. In this way, the user can easily identify the measurement target point that should be determined. If the X coordinate of the measurement target point is preliminarily determined, an input from the user may be limited to the Y coordinate, and any input on the X coordinate may be ignored. In this way, the user can concentrate on the adjustment of the Y coordinate. In a frame image, the X coordinate represents a horizontal (left to right) direction, whereas the Y coordinate represents a vertical (top to bottom) direction.

If the contours of the diaphragm and the lung field are unclear in the frame image displayed in the image display region 341a, the user should visually identify a small luminance gradient to specify the measurement target point. This may be supported by a function of appropriately enlarging the image to a desired size in response to a user operation. For example, an area containing the position specified through an operation of, for example, the mouse of the operating unit 33 on the frame image displayed in the image display region 341a may be enlarged. Alternatively, for the same purpose, an edge enhanced image acquired by carrying out edge enhancement on a frame image in response to a user operation may be displayed in the image display region 341a. Edge enhancement includes processing carried out with, for example, a first derivation filter, a Sobel filter, or a Prewitt filter. In this way, the user can easily identify the measurement target point to be specified.

During specification of the position of the measurement target point by the operating unit 33, the cursor sometimes acts as a visual interference that causes a slight misalignment of the measurement target point to be specified and the actual position of the diaphragm on the image. The cursor is translucent to prevent such interference. Alternatively, the portion indicating the position of the cursor may have a shape that is easily visible, such as a cross. The color of the cursor may be varied on the basis of luminance information on the image. For example, the cursor may have a bright color in an area having low luminance. This supports the visualization of the position of the diaphragm and the cursor. The description above is based on a GUI that determines the position of the measurement target point immediately after the user clicks on the position with the operating unit 33, such as a mouse. Alternatively, the position selected by clicking may be displayed as a temporary position of the measurement target point. This temporary position of the measurement target point may move in accordance with the mouse position during dragging. Upon release of the mouse, the position where the mouse was released may be determined as the measurement target point.

Manual specification of the measurement target point by the user may cause false specification of the measurement target point. It is preferred that, as a corrector in such a case, there may be provided a function that automatically adjusts the position of the measurement target point or a function that manually corrects or deletes the measurement target point.

As the function that automatically adjusts the position of the measurement target point, the measurement target point is moved to an area having a large luminance gradient near the measurement target point. An area having a large luminance gradient is equivalent to an area in an edge enhanced image having large pixel values. If an area having a large luminance gradient cannot be found near the specified measurement target point, the specification is determined to be an input error. Thus, the error is notified to promote re-entering of the measurement target point and receive specification of measurement target point again. An error can be notified by, for example, changing the color of the measurement target point determined to be an error, displaying the content of the error in text, changing the shape and symbol of the measurement target point determined to be an error, flashing the measurement target point determined to be an error, or outputting sound. The error may be notified in a case where a measurement target point is specified outside an area preliminarily determined to be the range where the diaphragm possibly moves.

As the function that manually adjusts the measurement target point, it is desirable that, in a step after the specification of the approximate position of the measurement target point by the mouse of the operating unit 33, fine adjustment can be made on the specified position of the measurement target point by moving the measurement target point by short distances in response to the operation of, for example, mouse wheel. It should be noted that the position of the measurement target point may be moved by a large distance in response to a dragging operation of the mouse, to make major adjustments. It is preferred to vary the size and/or color and perform flashing of the measurement target point during adjustment. It is further preferred to provide a function that deletes the specified measurement target point through operation of, for example, a delete key, to perform specification again.

It is preferred to provide a function that prompts the user to accept the finally specified measurement target point including the correction, to carry out measurement at the measurement target point desired by the user. For example, an acceptance button may be disposed near the image display region 341a and pressed through the operating unit 33, or a predetermined acceptance action, such as double-clicking of a specified point, may be carried out. In response, the controller 31 determines the specified point to be the measurement target point.

The measurement target point may be specified for a plurality of structures. For example, by specifying the points of the diaphragm and the apexes of the lungs, the chronological variation in the size of the lungs can be observed.

Alternatively, a plurality of measurement target points may be specified for a single structure (the diaphragm in this case). Alternatively, the measurement target point may be specified by a line. In this way, route candidates, which are described below, can be limited on the basis of the spatial relation among a plurality of points, to enhance the stability of the position measurement. The measurement target point is specified by a plurality of points or a line and the positions of the plurality of points and the line are tracked and measured, to identify the variation in the shape of the diaphragm. Thus, characteristic deformation of the diaphragm inherent in diseases such as COPD can be identified, to achieve early detection in patients having indications of COPD.

The above function in a case of specification by a single point is also included in a case of specifying the measurement target point by a plurality of points or a line.

In the case where a plurality of measurement target points is specified, the number of points to be specified should not be especially limited. The measurement target point may be specified in a plurality of frame images. In this way, the variation in the shape of the diaphragm can be observed. For example, the costophrenic angle is two-dimensionally tracked and the points specified in the diaphragm are tracked in the Y direction. These results of tracking may be combined to obtain tracking result for the position of the entire contour of the diaphragm. Such a process can extract the two-dimensional positional movement of the contour of the diaphragm.

It is preferred that the initial setting input screen 341 have the following functions to allow specification of a line as the measurement target point (specification of the position of the diaphragm in the form of a line). For example, a basic function allows the user to draw a line at a desired position in the image display region 341a with a pointing device of the operating unit 33. However, drawing a precise line with merely this function is difficult and a burden to the user. Thus, the initial setting input screen 341 further has a function of automatic interpolation (interpolation) between two or more points specified by the operating unit 33 on the diaphragm, to allow the user to easily specify a line of the diaphragm. The range between the specified points can be automatically interpolated through, for example, dynamic programming, template matching or tracing of large luminance gradient sites. Alternatively, the range between the specified points may be automatically interpolated by a segment line, and then the points may be adjusted to large luminance gradient sites near the segment line, to determine the exact line of the diaphragm.

Alternatively, the initial setting input screen 341 may have a function that allows the user to operate the operating unit 33 to surround an area including the diaphragm with a figure and detect a large luminance gradient site in the figure to be the diaphragm. The figure is, for example, a rectangle, a circle, or any other shape outlined using a pen tool. Such functions allow the user to easily specify the line of the diaphragm.

If the line of the diaphragm is falsely specified, the false line needs to be corrected. For example, in the case where the user specifies a plurality of points, when the user operates the operating unit 33 to specify another point between two specified points, the controller 31 performs detection of line segment again with the shortened distance between points. In this way, lines of the diaphragm can be specified more accurately. Alternatively, the falsely detected points may be surrounded with a rectangle using the operating unit 33 and deleted so that, when new points are specified in this area by the operating unit 33, the sections between the points may be automatically interpolated by the above-described method. In this way, a falsely detected group of points can be collectively deleted to perform correction.

The measurement target point having the same X coordinate may be specified with respect to a plurality of frame images. In this way, the positional information on the specified point can be used as a limitation condition when route candidates are to be limited or when the measurement results are to be corrected. For example, a limitation condition of the route candidates may be whether a route candidate passes near the specified point.

The initial setting input region 341b has an input field that receives input of information required for setting of the extraction range of the diaphragm position candidates, for example, the threshold of the speed (movement speed) or acceleration of the diaphragm, parameters such as the search direction in the template matching, as illustrated in FIG. 4. The diaphragm basically moves in the vertical direction, and the possible movement speed and acceleration are limited. Thus, in this embodiment, the extraction range of the diaphragm position candidates can be limited by the possible movement speed and acceleration of the diaphragm. The initial setting input region 341b receives input of the thresholds of the speed and the acceleration of the diaphragm. The movement of the diaphragm is basically in the vertical direction. Thus, the default search direction is the vertical direction. The user can specify any search direction desired by the user in the initial setting input region 341b. This embodiment will be described through an example in which the search direction is the vertical direction.

Upon pressing the enter button 341d in the initial setting input screen 341 by the operating unit 33, the content input from the initial setting input screen 341 is set in the RAM. The process then goes to Step S13.

It should be noted that default values of the parameters required for position measurement process may be preliminarily set without display of the initial setting input screen 341. Such a configuration can decrease the number of required operations and reduce the burden on the user.

In step S13, the variable n is determined to be 1 (step S13), and the n-th frame image is pre-processed (step S14).

In step S14, the n-th frame image is subjected to noise cancellation and gradation correction. Noise cancellation is carried out through filtering with, for example, a median filter, a moving average filter, or a Gaussian smoothing filter. Edge enhancement is then carried out. Edge enhancement is carried out through filtering with, for example, a first derivation filter, a Sobel filter, or a Prewitt filter. Such pre-processing carried out on a frame image removes noise information included in the frame image and enhances the diaphragm, to increase the accuracy of measurement of the diaphragm position.

It is then determined whether n=1 (step S15).

If n=1 (YES in step S15), a template image is prepared from the region around the measurement target point of the first frame image (step S16). For example, an area of w×h pixels (where w and h are positive integers) centered on the measurement target point in the first frame image is acquired to be a template image. The Y coordinate of the measurement target point is stored in the route storage unit 321 as the position of the diaphragm in the first frame (step S17). The process then goes to Step S22.

If n≠1 (NO in step S15), the n-th frame image is subjected to template matching using the template image prepared in step S16, to calculate correlation values (for example, cross-correlation coefficients) between the template image and the respective areas of w×h pixels centered on positions which are located within a predetermined range of the Y coordinates on the same X coordinate as the measurement target point (step S18; evaluation value calculator). The correlation values calculated in step S18 are evaluation values representing the similarity between the diaphragm and the positions along the Y coordinate.

It is preferred to calculate the evaluation values through normalized cross-correlation (NCC). Other calculation schemes include sum of squared difference (SSD), sum of absolute difference (SAD), and zero-means normalized cross-correlation (ZNCC).

The positions corresponding to the local maximum correlation values within the extraction range which is limited by the speed and acceleration set on the initial setting input screen 341 are extracted as diaphragm position candidates (step S19; position candidate extractor).

The positions corresponding to the local maximum correlation values are extracted to be the diaphragm position candidates instead of determining the position of the maximum correlation value to be the position of the diaphragm. Thus, the positions having relatively high correlation values in a spatial view can be determined as candidates even if the correlation values are not absolutely high values, and all of the positions corresponding to the diaphragm can be extracted as position candidates even under a condition that causes low absolute correlation values, such as overlapping with another large structure. Although the extraction range is one dimensional here, in a case of two-dimensional search, local maximum points may be extracted two dimensionally. In step S19, it is presumed that a plurality of diaphragm position candidates is extracted from at least one frame image.

The diaphragm position candidates to be extracted are limited to the positions corresponding to local maximum correlation values within the predetermined range of speed and acceleration of the diaphragm. Thus, it is possible to significantly remove the position candidates which are obviously not representing the diaphragm, and thus reduce the calculation load. The speed of the diaphragm is defined to be the movement amount of the diaphragm between frame images and is determined to be, for example, the difference of the Y coordinates of the positions of the diaphragm in one frame image and a frame image that is immediately previous of the one frame image. The acceleration of the diaphragm is defined to be a variation in movement amount of the diaphragm between frame images and is determined to be a difference between two consecutive speeds in time periods not overlapping each other (as described above). For example, the acceleration can be calculated by determining the difference between the above difference of Y coordinates and the difference between the Y coordinates of the position of the diaphragm in the frame image that is immediately previous of the one frame image and a frame image that is previous of the one frame image by two frames, the above difference being the difference of the Y coordinates of the positions of the diaphragm in the one frame image and the frame image that is immediately previous of the one frame image.

FIG. 5 is a schematic view of the extraction process of a diaphragm position candidate in step S19. The vertical axis of the graph in FIG. 5 represents the Y coordinate in the edge enhancement image on the right. In the case where local maximum points in the frame images captured at times T−1 and T−2, which are indicated by the circles in the drawing, are extracted to be diaphragm position candidates, a point in the frame image captured at time T within the predetermined range of the speed and acceleration (indicated by the circle) is extracted among the points having the local maximum correlation values to be the diaphragm position candidate.

It is preferred to preliminarily limit the search range of the template matching in step S18 on the basis of the speed and acceleration determined in the initial setting input screen 341 because calculation of correlation values corresponding to unnecessary ranges can be avoided and thus the calculation load can be reduced.

The position of the diaphragm in the first frame image corresponds to the position of the specified measurement target point, whereas a plurality of diaphragm position candidates may be extracted from the second and subsequent frame images. For example, if P diaphragm position candidates are extracted from the second frame image, P routes of the movement of the diaphragm are defined from the measurement target point in the first frame image to the respective P diaphragm position candidates in the second frame image. Diaphragm position candidates from the third frame image are extracted by determining whether the speeds and accelerations of the local maximum points are within a predetermined range when the local maximum points obtained through template matching are added to the respective P route candidates, and determining the local maximum points having speeds and accelerations within the predetermined range to be diaphragm position candidates of the respective route candidates. Diaphragm position candidates of the respective route candidates are extracted from the subsequent frame images in a similar manner. If the ranges limited by the speed and acceleration overlap among the route candidates, the position candidates may be extracted collectively in only a single operation. This avoids redundant calculation.

The extraction range of the diaphragm position candidates described above is limited by the speed and acceleration of the diaphragm. Alternatively, the extraction range of the diaphragm position candidates may be limited by either one of the speed and the acceleration. Alternatively, the respiratory condition at the time of image capturing of an n-th frame (for example, expiratory interval, resting (maximal) expiratory position, inspiratory interval, and resting (maximal) inspiratory position) may be determined on the basis of the information on the respiratory condition added to the n-th frame image, and the diaphragm position candidates to be extracted may be limited on the basis of whether the direction of movement of the diaphragm matches the respiratory condition at the time of image capturing of the frame image. Such limitation schemes may be used alone or in combination.

Route candidates including the diaphragm position candidates in the n-th frame image are prepared and stored in the route storage unit 321 (step S20).

FIG. 6 illustrates an example route storage unit 321. The route storage unit 321 stores information on route candidates indicating the movement of the diaphragm by chronologically linking each of the diaphragm position candidates extracted from the 1st to N-th frame images. With reference to FIG. 6, the route storage unit 321 stores the identification information (for example, route 1, route 2, route 3 . . . ) on the route candidates in correlation with the Y coordinates of the diaphragm position candidates corresponding to the respective route candidates in the respective frame images. In FIG. 6, the character “Y21” represents the first diaphragm position candidate in the second frame image. The term “uncalculated” indicates that the diaphragm position candidate is not yet extracted. The information on whether a diaphragm position candidate is uncalculated may be stored separately for each frame image. The route storage unit 321 may have any other configuration than that illustrated in FIG. 6. For example, frames 1 to 3 in route 3 are identical to frames 1 to 3 in route 4. Thus, these frames may be compressed and stored (for example, the Y coordinates may be stored in the fields of route 3, and flags indicating their identity with route 3 may be stored in the fields of route 4). Not only “Y coordinates” of the diaphragm position candidates, the route storage unit 321 may also store “correlation values.”

In step S20, for a route candidate having a single extracted diaphragm position candidate, the Y coordinate of the diaphragm position candidate is additionally stored in the field of the n-th frame image in the route storage unit 321. For a route candidate having two or more extracted diaphragm position candidates, a row for storing a new route candidate is added and stored in the route storage unit 321. The content of the route candidate to be added is identical to that of the previous route candidate except for the diaphragm position candidate in the n-th frame image. Thus, the identical information on diaphragm position candidate can be copied and stored for the other frame images.

The route candidates stored in the route storage unit 321 are limited to fewer route candidates (step S21; route limiting unit).

There are cases where the correlation value of the diaphragm position with the template image does not have a maximum value in a local temporal view because of overlapping with other organs and such like. However, the correlation value of the diaphragm position with the template image often has a maximum correlation value in a global view in the time direction. Thus, the route candidates stored in the route storage unit 321 are limited to route candidates having high correlation values within the global temporal range. The route candidates may be limited after obtaining all route candidates for all frame images. Alternatively, the route candidates may be limited during sequential processing, as described in this embodiment. Limiting the number of route candidates during sequential processing reduces the calculation load and thus enhances the processing speed. This also significantly reduces the storage capacity used for storing the route candidates.

In specific, step S21 is carried out to limit the route candidates, for example, when the number of route candidates at a time of extraction of diaphragm position candidates in a predetermined frame image exceeds a predetermined number. The route candidates are limited by, for example, determining the sum or average of correlation values of the determined diaphragm position candidates with the template image calculated so far (hereinafter, referred to as “correlation values of diaphragm position candidates”) and selecting the route candidates having such sums or averages being a predetermined threshold or more, as illustrated in FIG. 7. Alternatively, an arbitrary number of route candidates may be selected in a descending order of the sums or averages of the correlation values of the diaphragm position candidates. Alternatively, when there are correlation values of the diaphragm position candidates which are higher than or equal to a predetermined threshold at the time point of one frame image, the route candidates may be limited to those including these diaphragm position candidates. Alternatively, an arbitrary number of route candidates having the most frame images having the maximum correlation values of diaphragm position candidates may be selected in a descending order.

Alternatively, the route candidates may be limited on the basis of the following: the number of frame images or rate of the number of frame images including diaphragm position candidates having correlation values higher than or equal to a predetermined threshold or lower than a predetermined threshold among all the determined diaphragm position candidates; the sum or average of the rankings which are obtained by comparing the correlation values of the diaphragm position candidates calculated from each same frame image among the determined route candidates; or the number of frame images or rate of frame images which have rankings higher than or equal to a predetermined threshold or lower than a predetermined threshold, the rankings being obtained by comparing the correlation values of the diaphragm position candidates calculated from each same frame image among the plurality of route candidates.

Alternatively, if a measurement target point is specified on a same X coordinate in a plurality of frame images, the limitation condition of the route candidates may be route candidates passing near each measurement target point.

An evaluation index for limiting the route candidates may be the amount and/or the direction of the movement of a diaphragm position candidate, in place of a correlation value. This is because the diaphragm vertically and cyclically moves within a predetermined range in response to respiratory movement and thus moves by a limited amount while moving without intermittent changes in direction. For example, a predetermined number of route candidates corresponding to diaphragm position candidates moving by a small distance may be selected in an ascending order. Alternatively, the route candidates may be limited on the basis of whether the movement of the diaphragm matches the respiratory movement. For example, the route candidates may be limited to those in which positive or negative speeds of the diaphragm continue for a predetermined number of frame images.

The processes of limiting the route candidates described above are all carried out on the information on all frame images after extraction of diaphragm position candidates. Alternatively, information on frame images after division of routes may be used. For example, in FIG. 7, information on the sixth and subsequent frames in which the route is divided are used. This allows observation of only the time range relevant to limitation of the route candidates, thereby achieving high distribution of information and increasing the accuracy of the limitation of the route candidates. This also reduces the calculation load and thus enhances the processing speed. In specific, information on consecutive frame images from the frame image in which the route is divided to the final frame image is used. Alternatively, only the information on consecutive frame images from the frame image in which the route is divided to the frame image in which the diaphragm position candidates converge may be used. Alternatively, information on the frame images before and after the frame image in which the route is divided may be added. This allows appropriate route candidates to be selected under consistent time before and after the division. For example, the addition of information on frame images corresponding to approximately one second allows determination of whether each route candidates represents a slow movement typical of the diaphragm or whether the change in direction of movement matches the respiratory movement.

If a plurality of measurement target points (a plurality of points) is specified in step S12, the route candidates can be limited on the basis of the spatial relation of the plurality of points in each frame image. For example, if the contour of the diaphragm estimated from the position candidates at the plurality of points is discontinuous, not smooth, or inconsistent with the estimated shape, the route candidates including the diaphragm position candidates causing such discontinuousness, nonsmoothness, or inconsistency are removed. Whether the contour of the diaphragm matches the contour estimated from the position candidates at the plurality of points can be determined on the basis of, for example, the sum of the differences from a curve approximation by a quadratic or cubic function. Alternatively, the route candidates may be limited on the basis of the spatial relation among the plurality of points in a plurality of frame images. Any site of the diaphragm moves along the same direction during expiration and inspiration. Thus, if the plurality of points in the plurality of frame images does not move in the same directions, the route candidates including diaphragm position candidates having movement different from the other diaphragm position candidates may be deleted. Alternatively, the route candidates may be limited on the basis of whether the Y-coordinate movement width of the plurality of points is larger when closer to the costophrenic angles (anterior side) and smaller when closer to the spine.

The route candidates may be limited using a single evaluation index or a combination of two or more evaluation indexes.

For example, if the sum of correlation values and the movement amount of the diaphragm position candidates are used as evaluation indexes, these may be independently used to limit the route candidates so as to keep the route candidates satisfying predetermined conditions involving these evaluation indexes. This even more decreases the number of route candidates. Alternatively, two evaluation indexes may be weighted with a weighting factor and combined to be used as a single evaluation index. In this way, a plurality of evaluation indexes can one-dimensionally limit the route candidates.

The route candidates are limited in step S21 only in the case of a plurality of route candidates. The route candidates are also limited in step S21 if the route candidates stored in the route storage unit 321 satisfy a predetermined condition (for example, the number of route candidates exceeding a predetermined value).

It is determined whether steps S14 to S21 are completed for all frame images (step S22).

If steps S14 to S21 are not completed for all frame images (NO in step S22), the variable n is incremented by one (step S23). The process then goes to step S14.

If steps S14 to S21 are completed for all frame images (YES in step S22), one of the route candidates is determined to be the movement route of the diaphragm indicting the movement of the diaphragm, and the diaphragm position candidates in the frame images of the determined route are determined to be the positions of the diaphragm in the frame images (step S24; route determiner).

The route is determined in step S24 in the same manner as in step S21 of limiting the route candidates, except that a single route candidate is selected instead of an arbitrary number of route candidates. In the case where information on the respiratory movement during capturing of a dynamic image is added to the dynamic image as additional information, it is preferred that the route candidates be limited on the basis of whether the movement of the diaphragm in each route candidate corresponds to the respiratory movement during capturing of the dynamic image. For example, in the case where information on the number of breaths is set as additional information of the dynamic image, it is preferred that the route be determined after limiting the route candidates to route candidates provided with additional information on the number of breaths matching the number of breaths estimated on the basis of the diaphragm position candidates for the entire image capturing operation.

The measurement results of the position of the diaphragm are displayed on the display unit 34 (step S25; result output unit).

FIG. 8 illustrates an example measurement result screen 342 displayed on the display unit 34 in step S25. With reference to FIG. 8, the measurement result screen 342 includes an image display region 342a displaying a dynamic image including dots at the diaphragm positions in the frame images; a graph 342b illustrating the diaphragm waveform in which the Y coordinates of the position of the diaphragm are plotted along the time axis; the validity of the measurement result 342c; an acceptance button 342d; a correction button 342e; and a retry button 342f. The vertical axis of the graph 342b may be represented with the actual pixel positions on the radiation detector 13.

Dots having any size or lines having any thickness may represent the position of the diaphragm in the dynamic image displayed in the image display region 342a. The shape of the dots may be circles or squares. The size and shape of the dots may be selected by the user through the operating unit 33 or may be predetermined by default. It is preferred to provide a function that varies the color of the dots depending on, for example, the speed and acceleration of the diaphragm in consideration of visual analysis of the characteristics of positional variation of the measurement target points, by the user.

In the graph 342b, the position of the diaphragm in each frame image may be represented by dots or a line connecting the dots. The dots representing the position of the diaphragm in the frame images may have any size. The dots may have any shape including circles and squares. The size and shape of the dots may be selected by the user through the operating unit 33 or may be predetermined by default.

The line representing the position of the diaphragm in each frame image is preferably a broken line or an approximated curve. Similar to the dots, the lines may have any thickness.

In the case where a plurality of measurement target points is simultaneously specified, the measurement results are collectively represented by a single graph. In such a case, it is preferred to have a function that the graph has a different color for each measurement target point. In the case where the position of the diaphragm is indicated by dots, the dots may have different shapes depending on the measurement target point, in place of difference colors.

It is preferred to provide a function that instantaneously displays the corresponding frame image in the image display region 342a in response to selection of a point on the graph 342b through the operating unit 33, to efficiently display frame images which are to be referred to by the user.

Detailed analysis of the variation in the position of the diaphragm by the user may require a numerical display of the coordinates of the position of the diaphragm, in place of a graphical display. Thus, it is preferred that the measurement result screen 342 displays a table of the coordinates of the position of the diaphragm in each frame image, besides the display in FIG. 8. It is preferred to provide a function that not only displays the coordinates of the position of the diaphragm in each frame image but also displays a color map having cells of the table filled with different colors depending on the speed and acceleration. In this way, the user can easily determine the characteristics of the movement of the diaphragm in each measurement target point during measurement of a plurality of measurement target points.

It is preferred to provide a function that instantaneously displays a corresponding frame image in the image display region 342a in response to a value in the table selected through the operating unit 33. This enables efficient display of frame images which are to be referred to by the user.

It is preferred that the numerical data on the coordinates of the position of the diaphragm in each frame image be output in an editable format, such as a CSV file, to an external unit, such as a computer, via the communication unit 35. In this way, the user can use the numerical data on the coordinates of the position of the diaphragm to quantitatively analyze the variation in the position of the diaphragm.

It is preferred to provide a function that calculates various feature amounts by the controller 31 on the basis of the measured position of the diaphragm and outputs these feature amounts to the screen of the display unit 34 and in the form of a file, such that the user can easily utilize the measurement results for diagnosis. Six examples of the feature amounts to be calculated are listed below.

### 1. Maximum Movement Amount of Diaphragm

The maximum movement amount of the diaphragm is the absolute difference between the maximum value and the minimum value of the Y coordinates of the position of the diaphragm.

### 2. Maximum Speed of Diaphragm

The maximum speed of the diaphragm is the maximum movement amount of the position of the diaphragm per unit time or unit frame. This value is basically calculated on the basis of the measurement results of all frame images in a dynamic image. Alternatively, the maximum speed of the diaphragm during expiration may be determined from the measurement results during expiration, or the maximum speed of the diaphragm during inspiration may be determined from the measurement results during inspiration.

### 3. Respiratory Time

The respiratory time refers to the expiratory time, the inspiratory time, or the time of one breath (the sum of expiratory and inspiratory times). The expiratory time is determined by measuring the duration of the upward movement of the Y coordinate of the diaphragm. The inspiratory time is determined by measuring the duration of the downward movement of the Y coordinate of the diaphragm. The time of one breath is determined by adding the expiratory and inspiratory times. The expiratory time and inspiratory time are compared by, for example, determining the difference between the expiratory time and inspiratory time or ratio of the expiratory time to the inspiratory time.

### 4. Variation in Respiration

During capturing of a dynamic image corresponding to a plurality of breaths, the movement amount and speed of the diaphragm and the respiration time may vary among breaths. Such a variation can be quantized by, for example, calculating the feature amounts involving the plurality of breaths, such as the maximum movement amount of the diaphragm, the maximum speed of the diaphragm, and the respiratory time, and determining the dispersion or standard deviation of the feature amounts and the difference between the maximum and minimum values of each feature amount. Alternatively, similar calculations may be conducted on the coordinates of the diaphragm at the maximal inspiratory position per breath and the maximal expiratory position per breath, in place of the movement amount of the diaphragm per breath. Alternatively, similar calculations may be conducted on the respiratory time, i.e., the expiratory and inspiratory times.

### 5. Maximum Distance Between Diaphragm and Predetermined Reference Point

The maximum distance between the diaphragm and a predetermined reference point is the distance between the position of the diaphragm and a reference point when the position of the diaphragm is furthest from the reference point. The reference point is preferably the apex of the lung. The reference point may be any other point, such as a clavicle, the intersection of the thorax and the clavicle, the hilum of the lung, or the tracheal bifurcation.

### 6. Normalized Maximum Movement Amount of Diaphragm and Normalized Maximum Speed of Diaphragm

Feature amounts, such as the maximum movement amount of the diaphragm and the maximum speed of the diaphragm, should be normalized among targets if the dynamic image is to be compared with a dynamic image of the chest area of another patient. Thus, the feature amounts, such as the maximum movement amount of the diaphragm and the maximum speed of the diaphragm, are divided by a value representing the unique dimensions of the target. A value that represents the unique dimensions of the target may be any value such as a feature amount of the maximum distance between the diaphragm and a predetermined reference point, height, area of the lung field, or the width of the thorax. The feature amount of the maximum speed of the diaphragm may be normalized within the diaphragmatic excursion of the target. In specific, the maximum speed of the diaphragm is divided by the maximum movement amount of the diaphragm.

The feature amounts determined from the measurement results of the position of the diaphragm may be output as numeric data, as well as a graphical display and a dynamic image. Display schemes of the numeric data are described below.

For example, in the case where only one feature amount is to be displayed, it is preferred to display a table of the feature amounts in the measurement result screen 342, together with the dynamic image and the graph 342b. If the feature amounts suggest a disease, the display of the feature amounts should be varied to indicate the severity of the disease suggested by the feature amounts. In particular, the feature amounts indicating a severe disease should be displayed in a distinctive manner. For example, the characters or cells in the table may be displayed in colors corresponding to three levels of the disease of “normal,” “suspicious,” and “abnormal”: a normal level is displayed in a cold color, such as blue or green; and a suspicious or abnormal level is displayed in a warm color, such as yellow or red. Similarly, the level closer to abnormality may be represented by the larger size or thickness of the characters. Such a display format is also used for display of graphs and dynamic images, as described below.

The feature amounts may be written in the graph 342b. The display scheme of writing only one feature amount on a graph will now be described.

In the case where the feature amount is to be written on the graph 342b, it is preferred to display the feature amount near the value used for the calculation of the corresponding feature amount in the graph 342b. For example, in the case where the feature amount of the maximum speed of the diaphragm is to be written on the graph 342b, the value is written near the time and position of the maximum speed of the diaphragm. Alternatively, the feature amount may be displayed at any other display position.

In the case where the feature amount is to be written on the dynamic image displayed in the image display region 342a, it is preferred to display the feature amount in an area without visual interference of the diaphragm, such as the upper right corner of the dynamic image, for example. However, the present invention is not limited to this. It is preferred that the display of the feature amount in the frame image corresponding to the feature amount be distinguished from that in other frame images. For example, the feature amount of the maximum speed of the diaphragm may be displayed in an increased size in the frame image corresponding to the maximum speed of the diaphragm. Alternatively, the color of the feature amount may be changed.

In the case where a plurality of feature amounts is to be displayed, the display scheme of the individual feature amounts is the same as the display scheme for one feature amount. Unfortunately, displaying the plurality of feature amounts decreases the visibility of the screen. Thus, the number of feature amounts to be displayed should be reduced. For example, only the feature amounts indicating a disease may be displayed. Alternatively, only the feature amounts to be watched by the user may be displayed. In such a case, there is provided a user interface that allows the user to select a desired feature amount through the operating unit 33. Alternatively, a feature amounts that is frequently used for diagnosis may be displayed as default and there may be provided a user interface that allows the user to vary the number of feature amounts to be displayed. Such display schemes are compatible with the display of the feature amounts in the form of numeric values, on the graph 342b and in the dynamic image displayed in the image display region 342a.

The term “good” indicating high validity or the term “poor” indicating low validity is displayed in the region displaying the validity of the measurement result 342c. Examples of low validity of measurement results include a single route selected from an enormous number (a predetermined number or more) of prepared route candidates; a determined route including only a small number (less than a predetermined number) of diaphragm position candidates having high correlation values (higher than or equal to a predetermined threshold); and a determined route candidate including a large number (more than or equal to a predetermined number) of diaphragm position candidates having low correlation values (lower than a predetermined threshold). The preparation of an enormous number of route candidates suggests that the position of the diaphragm in many frame images cannot be determined in a single frame. This may lead to selection of a false route in step S24. A correlation value represents the similarity between the diaphragm region including the measurement target point and the template image. Thus, a route including many frame images having low correlation values is less likely to represent the accurate position of the diaphragm. Thus, in such a case, the low validity of the measurement result should be notified as an alarm to prompt the user to carefully observe the measurement results. In the case of low validity of the measurement result, the term “poor” is displayed. The specific reason of the low validity may be displayed, such as an enormous number of route candidates.

The measurement result screen 342 includes an user interface including an acceptance button 342d, a correction button 342e, and a retry button 342f, to input the final decision of the user on acceptance or correction of the measurement result or retry of the measurement. Pressing the correction button 342e causes a result correction screen 343 for correction of the measurement results to be displayed. Pressing the retry button 342f causes the initial setting input screen 341 to be displayed to retry the measurement.

It is determined whether the acceptance button 342d (or an acceptance button 343d or 344d, described below) of the measurement result screen 342 is pressed through the operating unit 33 (step S26).

If the acceptance button 342d (or the acceptance button 343d or 344d, described below) is not pressed through the operating unit 33 (NO in step S26), it is determined whether the correction button 342e (or a correction button 343e or 344e, described below) is pressed through the operating unit 33 (step S27).

If the correction button 342e (or the correction button 343e or 344e, described below) is pressed (YES in step S27), the result correction screen is displayed in the display unit 34 (step S28), and the measurement result is corrected in accordance with the operation of the operating unit 33 (step S29). The process then goes to step S26.

In one possible correction method of the measurement result, the user may manually move (for example, drag) the dot at a false position to the correct position in every frame image of the dynamic image through operation of the operating unit 33. The user however cannot manually correct all the false positions in frame images if the number of frame images including false positions is large. Thus, it is preferred to provide a function that switches the frame image (including a mark, such as a dot, representing the determined position of the diaphragm) of the dynamic image displayed on the result correction screen in response to a user operation and automatically corrects the frame images before and after a frame image manually corrected (dragged) by the user operating the operating unit 33 with reference to information on the position of the diaphragm in the manually corrected frame image. An example of such automatic correction includes interpolation between frame images based on the average of the position of the diaphragm in the manually corrected frame image and the positions of the diaphragm in the frame images before and after the manually corrected frame image.

As described above, a single route is selected from the plurality of route candidates. Unfortunately, the selected route may differ from the route desired by the user. In such a case, in order to enable the user to easily select an appropriate route, the plurality of route candidates may be displayed before selection of the single route and a user interface (for example, the result correction screen 343) may be displayed on the display unit 34, to allow the user to select the most appropriate route candidate among the displayed route candidates.

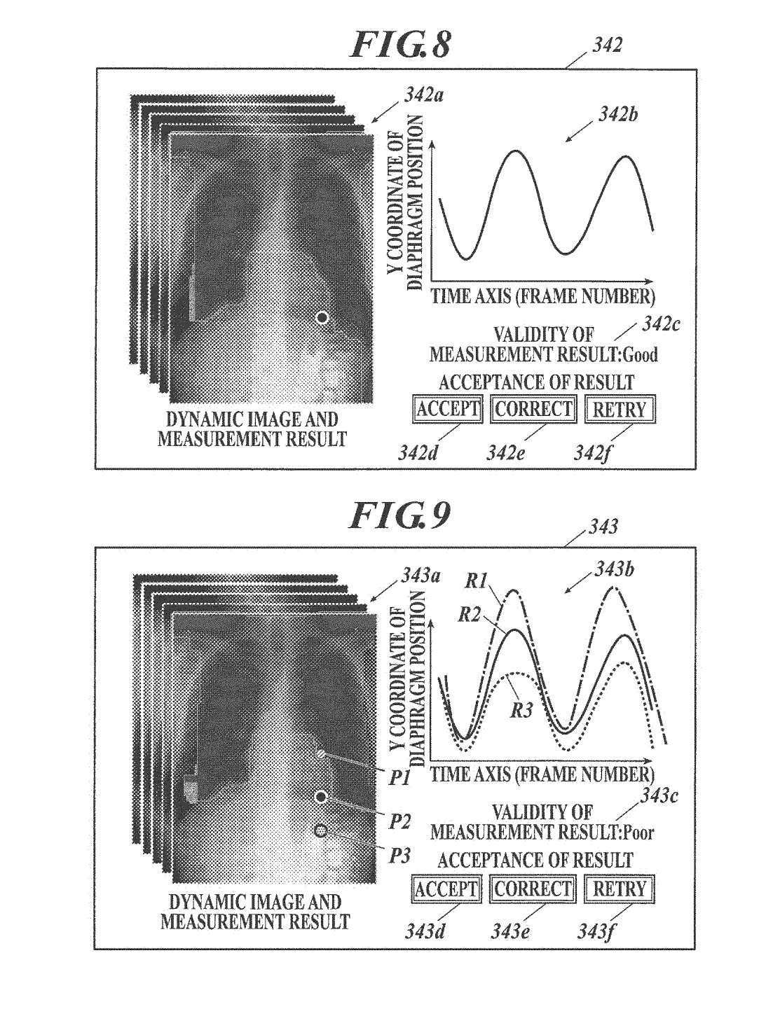

FIG. 9 illustrates an example of the result correction screen 343 described above. With reference to FIG. 9, the result correction screen 343 includes an image display region 343a, a graph 343b, the validity of the measurement result 343c, an acceptance button 343d, a correction button 343e, and a retry button 343f. The image display region 343a displays diaphragm position candidates P (three diaphragm position candidates P1 to P3 in FIG. 9) in a plurality of route candidates before determination of a single route, as illustrated in FIG. 9. The graph 343b illustrates the temporal variation R in the Y coordinate of the diaphragm position candidates in the route candidates (R1 to R3 in FIG. 9). In response to selection of one of the diaphragm position candidates P or one of the route candidates R through the operating unit 33, the route candidate corresponding to the selected point P or the selected route candidate R is determined to be the route of the diaphragm.

Alternatively, there may be displayed a user interface (for example, a result correction screen 344) capable of receiving input of an updated measurement target point.

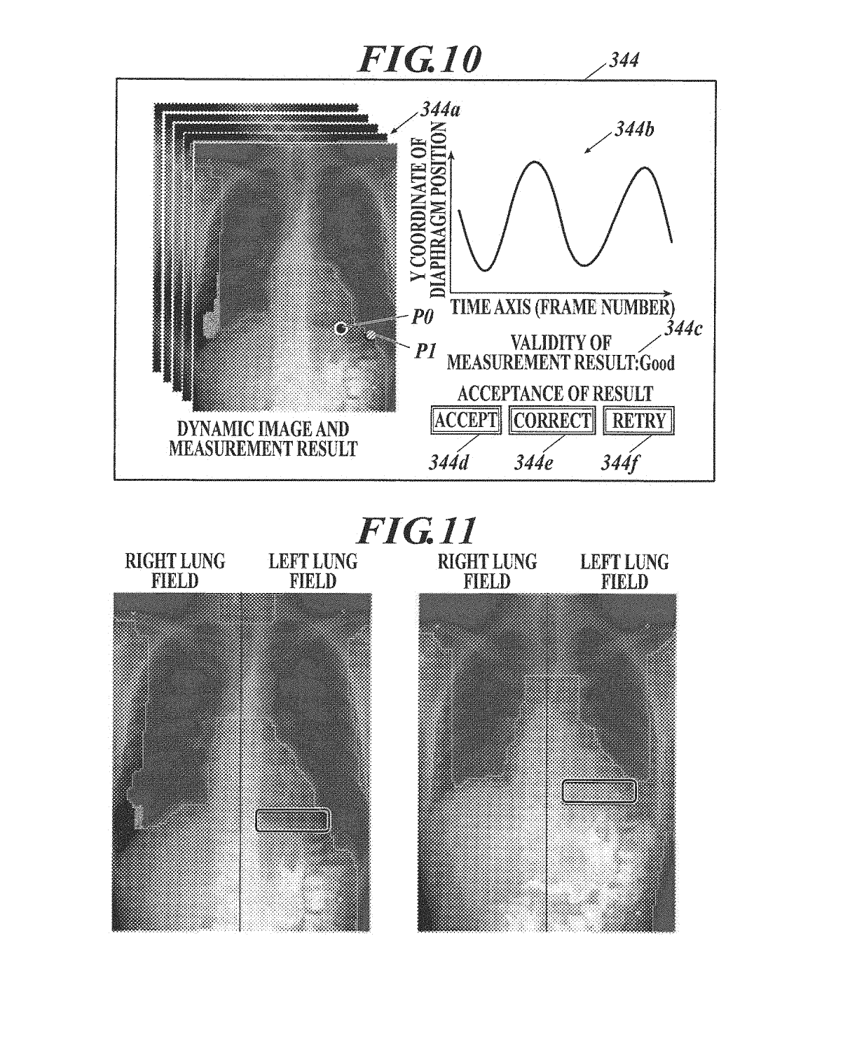

FIG. 10 illustrates an example result correction screen 344. With reference to FIG. 10, the result correction screen 344 includes an image display region 344a, a graph 344b, validity of the measurement result 344c, an acceptance button 344d, a correction button 344e, and a retry button 344f.

For example, the image display region 344a first displays the frame image including the specified measurement target point (referred to as the measurement target point P0), as illustrated in FIG. 10. Upon selection of an updated measurement target point P1 near the measurement target point P0 through the operating unit 33, the controller 31 calculates the movement route of the diaphragm on the basis of the measurement target point P1. For each frame image, the route having the highest validity is selected among the preliminarily determined route candidates on the basis of positional relation between the route candidates determined on the basis of the measurement target point P0 and the movement route of the diaphragm determined on the basis of the measurement target point P1. Alternatively, another frame image different from the frame image including the measurement target point P0 may be displayed in the image display region 344a to receive input of a measurement target point having an X coordinate identical to that of the measurement target point P0, and limit the route candidates to those passing through the updated measurement target point. In this way, routes can be accurately determined.

In step S27, if the correction button 342e (or after-mentioned correction button 343e or 344e) is not pressed (NO in step S27) and the retry button 342f (or after-mentioned retry button 343f or 344f) is pressed (YES in step S28), the process goes to step S12 to cause the initial setting input screen 341 to be displayed on the display unit 34 and retry the position measurement process.

If the acceptance button 342d (343d or 344d) is pressed through the operating unit 33 (YES in step S26) and the position measurement result is stored in connection with the dynamic image stored in the memory unit 32 (step S31), the position measurement process ends.

As described above, the controller 31 of the diagnostic console 3 calculates evaluation values representing the similarity between the diaphragm and the positions in predetermined ranges of the frame images of a dynamic image of the chest area; extracts at least one diaphragm position candidate from each of the frame images on the basis of the calculated evaluation values; and stores route candidates in the route storage unit 321, each route candidate being prepared by chronologically linking each diaphragm position candidate extracted from each frame image. One route candidate is selected among the route candidates stored in the route storage unit 321 to be the movement route of the diaphragm. The diaphragm position candidates in the selected movement route are determined to be the position of the diaphragm in the respective frame images.

Thus, even if the evaluation value of the actual position of the diaphragm is not a maximum value in a local temporal view because of, for example, overlapping with other structures, diaphragm position candidates can be extracted and chronologically linked to each other to determine the position of the diaphragm within a global temporal range. Thus, even if the contour (outline) of the diaphragm is unclear in a dynamic image of the chest area captured without a marker, the position of the diaphragm in the frame images can be accurately measured.

For example, the controller 31 extracts the positions at which the calculated evaluation values are the local maximums from the respective frame images to be diaphragm position candidates. Thus, the positions at which the absolute evaluation values are low but the relative evaluation values are high in a spatial view can be determined to be diaphragm position candidates. This allows all diaphragm position candidates to be extracted even under a condition in which the absolute evaluation values are low in a certain spatial range, such as in a case of overlapping with other structures.

For example, the controller 31 limits the extraction range of the diaphragm position candidates on the basis of the movement speed and/or acceleration of the diaphragm. This reduces extraction of diaphragm position candidates that cannot be the position of the diaphragm. This can also reduce the calculation load.

For example, the controller 31 limits the diaphragm position candidates to be extracted from the frame images on the basis of whether the direction of movement of the diaphragm matches the respiratory condition at the time of capturing of the frame images. This reduces extraction of diaphragm position candidates that cannot be the position of the diaphragm. This can also reduce the calculation load.

For example, the controller 31 determines the movement route of the diaphragm on the basis of the evaluation values calculated for the respective diaphragm position candidates included in each route candidate stored in the route storage unit 321. For example, the controller 31 determines the movement route of the diaphragm based on the sum or average of evaluation values of diaphragm position candidates in the route candidates, the number or rate of frame images including the diaphragm position candidates having evaluation values higher than or equal to or lower than a predetermined threshold in the route candidates, the sum or average of rankings of the evaluation values of the diaphragm position candidates calculated from the same frame image in the route candidates, or the number or rate of frame images in which rankings of the evaluation values of the diaphragm position candidates calculated from the same frame image in the route candidates is higher than or equal to a predetermined threshold or lower than a predetermined threshold, to accurately determine the movement route of the diaphragm.

For example, the controller 31 determines the movement route of the diaphragm on the basis of the amount and/or the direction of the movement of the diaphragm in the route candidates stored in the route storage unit 321, to accurately determine the movement route of the diaphragm on the basis of the estimated cyclic movement of the diaphragm.

The controller 31 causes the initial setting input screen to be displayed on the display unit 34 to allow the user to input initial setting for measurement of the diaphragm position. In this way, the user can input setting information regarding the measurement of the diaphragm position.

The controller 31 outputs the measurement result of the position of the diaphragm, to provide the measurement result of the position of the diaphragm to the user.

The controller 31 limits the plurality of route candidates stored in the route storage unit 321 to fewer route candidates, to reduce the storage capacity of the route storage unit 321.

For example, the controller 31 limits the route candidates on the basis of the evaluation values calculated for the respective diaphragm position candidates in the route candidates stored in the route storage unit 321. For example, the evaluation value is significantly high for a diaphragm position candidate matching the position of the diaphragm in a frame image not overlapping with other structures. Thus, the controller 31 limits the route candidates to those including diaphragm position candidates having evaluation values higher than or equal to a predetermined threshold. This omits route candidates that cannot be the route (the route candidates that do not pass through the diaphragm position candidates certainly matching the position of the diaphragm).

For example, the controller 31 limits the route candidates on the basis of the amount and/or the direction of the movement of the diaphragm in the route candidates stored in the route storage unit 321, to omit the route candidates that cannot represent the movement of the diaphragm.

For example, the controller 31 limits the route candidates on the basis of whether the movement of the diaphragm in the route candidates correspond to the respiratory movement during capturing of the dynamic image, to omit the route candidates that represent movement of the diaphragm not corresponding to the respiratory movement during capturing of the dynamic image.

The embodiments of the position measurement system described above are example and should not limit the scope of the present invention.

For example, in the embodiments described above, the target site is the chest area. Alternatively, the dynamic image may be of any other site.

In the embodiments described above, the position of the diaphragm is measured. Alternatively, the present invention may be applied to other structures, such as the heart wall, ribs, and blood vessels. The search direction of the position should be adjusted depending on the structure. For example, in the case where the target structure is the heart wall, it is preferred to search the horizontal direction (X direction). In the case where the target is a rib or a blood vessel, it is preferred to search the Y direction, which is the same as that in the search of the diaphragm. Besides the Y and X direction, the search direction may be a diagonal direction depending on the structure. Alternatively, a two-dimensional change in the position may be measured.

In the embodiments described above, diaphragm position candidates are extracted in chronological order from the first frame image. Alternatively, the diaphragm position candidates may be extracted in chronological and reverse chronological order from a reference frame image. For example, in the case where the seventh frame image among the first to fifteenth frame images is selected to be a reference frame image, diaphragm position candidates are extracted in reverse chronological order from the seventh to the first frame images and in chronological order from the seventh to the fifteenth frame images.

In the embodiments described above, the position of the diaphragm in all frame images of the dynamic image is measured. Alternatively, a range of the frame images of the dynamic image may be specified and position measurement may be carried out within the specified range.

In the embodiments described above, the user sets thresholds for the speed and acceleration of the diaphragm. Alternatively, the thresholds may be automatically set by the controller 31. In the embodiments described above, upper limits of the speed and acceleration are set. Not only the upper limits, but also lower limits may be set.

The thresholds of the speed and acceleration of the diaphragm can be automatically set as described below.

For example, the maximum value of the range of the speeds and accelerations calculated from the movement of the diaphragm in various dynamic images of the chest area is determined to be the upper threshold, whereas the minimum value is determined to be the lower threshold. Alternatively, a range of the speeds and accelerations calculated from the movement of the diaphragm in various dynamic images of the chest area may be provided with a margin to be the range of possible speeds and accelerations of the diaphragm, and the maximum and minimum values of this range may be determined to be the upper and lower thresholds, respectively. These thresholds can be updated when the range of the speeds and accelerations is updated through collection of dynamic images, to achieve more highly accurate measurement result of the diaphragm.

In the embodiments described above, the area of the w×h pixels centered on the measurement target point selected by the user in a dynamic image is determined to be a template image. Alternatively, the template image may be a geometric figure outlining the diaphragm, an image of a diaphragm extracted from sample data, or an image of a typical diaphragm prepared from many X-ray images.

In the embodiments described above, evaluation values representing the similarity between positions within a predetermined range in the frame images of the dynamic image and the diaphragm (a predetermined structure) are determined through template matching. The evaluation values may be determined through any other scheme. For example, the ratio of white pixels to black pixels in a digitized area of w×h pixels centered on a measurement target point is determined to be a reference value representing the characteristics of the diaphragm, and the ratio of white pixels to black pixels at each position to be searched for the diaphragm in a predetermined range (an area of w×h pixels centered on each pixel) is determined. The difference between each ratio and the reference value may be determined to be an evaluation value representing the similarity of each position and the actual diaphragm.

In the embodiments described above, the route candidates are sequentially limited. Alternatively, the limiting of route candidates can be omitted, for example, if the route storage unit 321 has large storage capacity.

In the description above, the program according to the present invention is stored on a computer readable medium, such as a hard disk or a non-volatile semiconductor memory. Alternatively, any other computer readable medium may be used. Other computer readable media include a portable recording medium, such as a CD-ROM. Carrier waves may also be applied to the present invention as a medium that provides data of the program according to the present invention via a communication line.

The detailed configuration and operation of the components of the position measurement system 100 according to the embodiments described above may be appropriately modified without departing from the scope of the present invention.

Although embodiments of the present invention have been described and illustrated in detail, it is clearly understood that the same is by way of illustration and example only and not limitation, the scope of the present invention should be interpreted by terms of the appended claims.

Japanese Patent Application No. 2016-208447 filed on Oct. 25, 2016, including description, claims, drawings, and abstract the entire disclosure is incorporated herein by reference in its entirety.

What is claimed is:

1. A dynamic image processor which measures a position of a predetermined structure in a plurality of frame images obtained by emitting radiation to a subject to perform dynamic imaging, the dynamic image processor comprising a hardware processor which calculates an evaluation value indicating similarity with respect to the structure for each position in a predetermined range in each of the plurality of frame images, extracts at least one position candidate of the structure from each of the plurality of frame images based on the calculated evaluation value, extracts a plurality of position candidates of the structure from at least one of the frame images, stores a plurality of route candidates in a storage, each of the route candidates being obtained by chronologically linking the position candidate of the structure extracted from each of the plurality of frame images to be a route candidate of movement of the structure, determines one of the plurality of route candidates stored in the storage as a movement route of the structure, and determines the position candidate of the structure included in the determined route as the position of the structure in each of the plurality of frame images.

2. The dynamic image processor according to claim 1, wherein the hardware processor extracts a position for which the calculated evaluation value is a local maximum as the position candidate of the structure in each of the plurality of frame images.

3. The dynamic image processor according to claim 2, wherein the hardware processor limits an extraction range of the position candidate of the structure based on a speed and/or an acceleration of the movement of the structure.

4. The dynamic image processor according to claim 2, wherein the hardware processor limits the position candidate of the structure to be extracted based on whether a direction of the movement of the structure matches a respiratory condition when each of the plurality of frame images is captured.

5. The dynamic image processor according to claim 1, wherein the hardware processor determines the movement route of the structure based on the evaluation value which is calculated for each of the position candidates of the structure included in each of the route candidates stored in the storage.

6. The dynamic image processor according to claim 5, wherein the hardware processor determines the movement route of the structure based on: a sum or an average of evaluation values of the position candidates of the structure in each of the route candidates; a number of a frame image or a rate of the number of the frame in each of the route candidates, the frame image including a position candidate of the structure which has the evaluation value higher than or equal to a predetermined threshold or lower than a predetermined threshold; a sum or an average of a ranking in each of the route candidates, the ranking being obtained by comparing the evaluation values of the position candidates of the structure calculated from a same frame image among the plurality of route candidates; or a number of a frame image or a rate of the frame image in each of the route candidates, the frame image having the ranking higher than or equal to a predetermine threshold or lower than a predetermined threshold.

7. The dynamic image processor according to claim 1, wherein the hardware processor determines the movement route of the structure based on an amount and/or a direction of the movement of the structure in each of the route candidates stored in the storage.

8. The dynamic image processor according to claim 1, further comprising an operating unit for a user to input initial setting for measuring the position of the structure.

9. The dynamic image processor according to claim 1, further comprising an output unit which outputs a measurement result of the position of the structure.

10. The dynamic image processor according to claim 1, wherein the hardware processor limits the plurality of route candidates stored in the storage to fewer route candidates, and determines one of the limited route candidates as the movement route of the structure.

11. The dynamic image processor according to claim 10, wherein the hardware processor limits the route candidates based on the evaluation value calculated for each of the position candidates of the structure included in each of the route candidates stored in the storage.

12. The dynamic image processor according to claim 11, wherein, when a position candidate of the structure has the evaluation value higher than or equal to a predetermined threshold, the hardware processor limits the route candidates to a route candidate which includes the position candidate.

13. The dynamic image processor according to claim 10, wherein the hardware processor limits the route candidates based on an amount and/or a direction of the movement of the structure in each of the route candidates stored in the storage.

14. The dynamic image processor according to claim 10, wherein the hardware processor limits the route candidates based on whether movement of the structure in each of the route candidates corresponds to a respiratory movement when the dynamic imaging is performed.

15. The dynamic image processor according to claim 1, wherein the predetermined structure is a diaphragm, a heart wall, a rib or a blood vessel.

16. The dynamic image processor according to claim 1, wherein the hardware processor calculates the evaluation value by template matching.
